(19) United States
(12) Patent Application Publication
(10) Pub. No.: US 2024/0269050 A1
(43) Pub. Date: Aug. 15, 2024

(54) ELECTROPORATION PROCESS FOR DELIVERING A COMPOSITION COMPRISING AT LEAST ONE HYDROXY ACID COMPOUND

(71) Applicant: L'OREAL, Paris (FR)

(72) Inventors: Thi Hong Lien PLANARD-LUONG, Chevilly Larue (FR); Patrycja NZOUNZA, Chevilly Larue (FR); Veronique BURNIER, Chevilly Larue (FR)

(21) Appl. No.: 18/564,784

(22) PCT Filed: May 27, 2022
(86) PCT No.: PCT/EP2022/064416

§ 371 Date: Nov. 28, 2023

(30) Foreign Application Priority Data
May 28, 2021 (FR) FR2105584

(51) Int. Cl. A61K 8/365; A61N 1/32; A61Q 19/08

(52) U.S. Cl. CPC A61K 8/365; A61N 1/327; A61N 1/328; A61Q 19/08; A61K 2800/83; A61K 2800/87

(57) ABSTRACT

The present invention relates to an electroporation process for delivering a composition through human keratin materials, the composition comprising at least one hydroxy acid compound, the electroporation process involving exposing the composition in contact with said keratin materials to pulse trains of a pulsed electric current, the pulsed electric current being provided by an electroporation device having at least one electrode and at least one counterelectrode, the pulse trains of the pulsed electric current repeating at a frequency of between 0.1 Hz and 1 kHz, the pulse trains each having a duration (Tₒₙ) of between 0.001 s and 2 s, and the pulsed electric current having a voltage amplitude ranging from 20 V to 250 V and a current ranging from 0.1 mA to 10 mA.

### TECHNICAL FIELD

The present invention relates to the field of caring for keratin materials, notably the skin. For the purposes of the present invention, the term “keratin materials” notably denotes the skin, the lips and/or the eyelashes, in particular the skin and/or the lips, and preferably the skin of the body and/or the face, and more preferentially of the face.

Nowadays, a very large number of treatments exist, involving or not involving cosmetic surgery, for enhancing the appearance of human beings. These treatments, which will be referred to hereinbelow as treatments for aesthetic purposes, may act in many ways, and may consist, for example, in effacing or masking certain skin imperfections, or alternatively attenuating or even effacing the signs of ageing.

Non-limiting illustrations of such treatments that may notably be mentioned are chemical peeling operations.

The term “peeling” conventionally denotes a treatment which consists in topically bringing about the destruction of the cell layers forming the epidermis, to a variable depth depending on the intensity of the peeling, so as to induce exfoliation of these layers, followed by their regeneration, i.e. their replacement with new cell layers. Conventional chemical peeling operations thus revitalize the surface of the skin by creating a uniform and controlled excretion of the skin cells.

Peeling operations advantageously make it possible to rejuvenate and retexturize the skin and to remove a certain number of imperfections present on the surface of the skin, such as acne, sunlight-mediated damage, pigmentation marks, wrinkles and fine lines, superficial scars, mattness, etc.

### PRIOR ART

Chemical peeling operations can be performed at home or by professionals, and often comprise the same active ingredients, such as α-hydroxy acids.

However, home peeling operations are less powerful and thus less efficient than peeling operations administered by professionals, notably since they can only affect the uppermost layers of the skin.

As a result, home peeling operations give results that are often barely perceptible, usually requiring several applications before appreciable effects are obtained.

Moreover, it is known that the application of an electric current to the skin can promote the penetration of an active agent. It is thus known practice to treat human keratin materials with the aid of electroporation devices. Electroporation allows the diffusion of active agents through the skin by means of electrical stimulation in a non-invasive manner.

Electroporation renders the surface of the skin permeable, allowing active agents to pass by diffusion, or even by electrophoresis when they are charged.

Patent applications US 2010/255079, US 2012/065575, US 2013/345307, US 2013/345661, WO 2010/112708 or WO 2008/045272 describe devices which combine an electric current with cosmetic active agents. However, these devices are occasionally not sufficiently efficient, notably as regards increasing the performance of home peeling formulations.

### DISCLOSURE OF THE INVENTION

A need thus remains to further improve the processes which promote the penetration of active agents through keratin materials so as to increase the efficiency of electroporation, and to benefit from a process notably for the cosmetic or dermatological treatment of keratin materials, making it possible to enhance the performance and to accelerate the action of peeling formulations by increasing the supply of peeling active principles inside the live part of the keratin materials, while at the same time being able to be used with comfort and in total safety.

There is in particular interest for home peeling operations that are virtually as efficient and powerful as peeling operations performed by professionals, so as to target deeper levels of the dermis at home and notably to treat moderate to severe wrinkles, damage caused by sunlight and pigmentation, and which give results that become apparent immediately or a short time after a single treatment, painlessly and without any muscle contraction induced by the electroporation.

There is a need to increase the cutaneous release of active agents in a targeted manner inside the live parts of keratin materials, and their bioavailability, so as to have access to efficient home peeling operations.

The invention is directed towards meeting all or some of these needs.

### SUMMARY OF THE INVENTION

Thus, according to a first of its aspects, the present invention relates to an electroporation process for delivering a composition through human keratin materials, the composition comprising at least one hydroxy acid compound, the electroporation process comprising the exposure of the composition in contact with said keratin materials to pulse trains of a pulsed electric current, the pulsed electric current being provided by an electroporation device having at least one electrode and at least one counterelectrode, the pulse trains of the pulsed electric current repeating at a frequency (fₜᵣₐᵢₙ) of between 0.1 Hz and 1 kHz, the pulse trains each having a duration (Tₒₙ) of between 0.001 s and 2 s, and the pulsed electric current having a voltage amplitude ranging from 20 V to 250 V, better still from 30 V to 250 V, and a current ranging from 0.1 mA to 10 mA.

The process according to the invention makes it possible to perform home peeling operations that are much more efficient than the conventional home peeling operations, with results which become apparent immediately or a short time after a single treatment.

Specifically, the cutaneous release of the hydroxy acid compounds inside the live parts of the keratin materials, and also their bioavailability, are increased in comparison with conventional home peeling operations, painlessly and without any muscle contraction induced by the electroporation.

According to a preferred embodiment, the process comprises topical application of the composition to the zone to be treated before applying the pulsed electric current and/or during said application.

### DETAILED DESCRIPTION

As indicated above, the electroporation process according to the invention comprises the exposure of a composition in contact with said keratin materials, preferably the skin, to electric pulse trains of a pulsed current, said current being delivered by an electroporation device having at least one electrode and at least one counterelectrode.

In accordance with the invention, the pulse trains of the pulsed current are repeated at a frequency (fₜᵣₐᵢₙ) of between 0.1 Hz and 1 kHz, the pulse trains each having a duration (Tₒₙ) of between 0.001 s and 2 s, and the current having a voltage amplitude ranging from 20 V to 250 V, and a current ranging from 0.1 mA to 10 mA.

### Electroporation Device

According to the invention, the pulsed electric current is provided by an electroporation device having at least one electrode and at least one counterelectrode.

Preferably, the pulse trains are repeated at a frequency fₜᵣₐᵢₙ which ranges from 0.1 Hz to 1 kHz, preferably from 0.3 Hz to 100 Hz, and more preferentially is equal to 0.4 Hz±20%. According to the invention, the pulsed electric current has a voltage amplitude ranging from 20 V to 250 V, better still from 30 V to 250 V, and even better still from 30 V to 100 V, and a current ranging from 0.1 mA to 10 mA. The amplitude is measured peak-to-peak and the current is the instantaneous current.

The frequency of the pulses within the pulse train is greater than the frequency at which the pulse trains repeat. The voltage can vary within the pulse train around a non-zero, notably negative, mean voltage amplitude value Vₘₑₐₙ, this mean voltage Vₘₑₐₙ preferably remaining substantially constant for the duration Tₒₙ of the pulse train. The voltage may notably vary within the pulse train at a frequency (fₚᵤₗₛₑ) of the pulses within the pulse train of between 5 kHz and 100 kHz, better still between 10 kHz and 50 kHz, for example between 30 and 40 kHz.

The amplitude deltaV of the voltage variation within the pulse train around the mean voltage value Vₘₑₐₙ corresponds, for example, to less than 50% of the value of this mean voltage (as an absolute value), or even to less than 30%.

Preferably, the pulsed electric current has a direct polarity (“DC current”), the polarity of the electrode notably being negative with respect to that of the counterelectrode.

The maximum current density per pulse may be less than or equal to 5×10⁻⁴ A.

The pulses may be unipolar, which means that the current travels in one direction. The pulse train may have a constant maximum amplitude Vₘₐₓ. The pulse train may have a variable amplitude, for example an amplitude which increases from a minimum amplitude, reaches the maximum amplitude, and then eventually decreases towards the minimum amplitude.

A pulse train may combine two or more pulse waveforms simultaneously or alternately. The application of the current may involve the generation of a pulsed current having sinusoidal waveforms, non-sinusoidal waveforms, periodic square waveforms, rectangular waveforms, sawtooth waveforms, spike waveforms, bell waveforms, trapezoidal waveforms, triangular waveforms, or combinations thereof.

Preferably, the pulsed electric current is of a general square or rectangular waveform. In particular, during a pulse train, the mean voltage may be equal to a negative Vₘₑₐₙ value, and between two pulse trains (during T_{off}) the voltage is zero.

A pulse train may comprise from 2 pulses to 15 000 pulses, each having a duration tₚᵤₗₛₑ.

Preferably, the pulse trains of the pulsed electric current each have a duration (Tₒₙ) ranging from 0.01 s to 3 s, preferably from 0.05 s to 2 s, and more preferentially from 0.5 s to 2 s.

According to a preferred embodiment, the pulse trains are separated by a pulse-free interval (T_{off}) ranging from 0.01 s to 3 s, preferably from 0.03 s to 2 s, and more preferentially from 0.5 s to 2 s.

According to a preferred embodiment, the pulsed electric current has a duty cycle (Tₒₙ/(Tₒₙ+T_{off})) ranging from 20% to 90%, preferably from 25% to 80%, where Tₒₙ denotes the duration of a pulse train and T_{off} denotes the interval between two successive pulse trains.

Preferably, the maximum voltage between the electrode and the counterelectrode in open circuit ranges from 20 V to 250 V.

### Electrodes

According to a preferred embodiment, the process comprises the step consisting in manually moving the electrode(s) of the electroporation device over the keratin materials while subjecting said keratin materials to said pulse trains of pulsed electric current.

According to another embodiment, the active electrode is stationary and applied at a single location on the keratin materials, for example through an adhesive.

The electrode(s) may have a rounded surface for contact with the keratin materials.

Preferably, the surface area of the electrode(s) in contact with the keratin materials ranges from 2 to 20 cm², and preferably from 2 to 10 cm².

Preferably, the counterelectrode is static and has a surface area in contact with the human keratin materials that is greater than that of the electrode, which is moved in contact with the keratin materials, the surface area of the counterelectrode preferably being more than 1.5 times that of the electrode.

The electroporation device may comprise at least one reservoir containing an effective amount of a composition according to the invention.

In particular, the device may comprise at least one electrode and one or more compositions stored in a reservoir such as a cavity, a gel, a laminate, a membrane, a porous structure, a matrix or a substrate.

More particularly, the device may comprise at least one active electrode electrically coupled to a reservoir containing a cosmetic composition according to the invention.

The reservoir may comprise any shape or material used for retaining a composition according to the invention.

The reservoir may comprise one or more ion-exchange membranes, semi-permeable membranes, porous membranes or gels that are capable of at least temporarily retaining a composition according to the invention.

The reservoir may comprise one or more cavities formed by a structure.

In particular, the electroporation device may comprise one or more ion-exchange membranes that may be positioned to serve as a polarity-selective barrier between the reservoir of composition according to the invention and a biological interface.

The reservoir may be a hollow imprint at the end of the electrode. As a variant, according to a particular embodiment, the reservoir is an absorbent material for containing the composition according to the invention.

In particular, the reservoir may be connected to a piston or to any other means for dispensing the composition. Thus, the active electrode may be provided with a type of ball applicator fed by a piston. The piston may be actuated during use of the electroporation device.

The electroporation device may also be an electroporation patch or an electroporation face mask.

Preferably, the electroporation device is hand-held.

### User Interface

The electroporation device may comprise a user interface.

The user interface may be intended for entering data regarding the types of waveforms to be applied as electrical stimuli. The user interface may comprise an alphanumeric keyboard and a display. The alphanumeric keyboard may be implemented as a touch screen.

In particular, the user interface may comprise directional arrow buttons and an input button for entering data into a memory.

The input of waveform parameters can be done by using text boxes and/or by using drop-down menus. Irrespective of how data is entered, the user interface may communicate a variety of prompts for the user to enter information.

The user interface may prompt the user to enter the duration of the treatment.

The user interface may prompt the user to enter the treatment zone.

The device may include a controller which runs logic routines to direct the user interface so as to present the user with appropriate prompts. The controller then uses the entered parameters to generate, via its electronics, including, but not limited to, the appropriate voltage based on the parameters entered by the user.

### Other Sensors

According to a particular embodiment of the electroporation process, the process comprises a step of measuring the skin temperature, the skin impedance and the pH of the composition.

Thus, the electroporation device may comprise at least a temperature sensor, an impedance sensor, and/or a pH sensor.

The application of the current profile can thus be reduced to a safety level when a value measured by one of the sensors exceeds a safety range or value.

According to a particular embodiment of the electroporation process, the process comprises a step of measuring the pH of the composition. When the measured pH exceeds a pH safety range, the application of the current profile is switched to a safety level, for example a safety level below 1 V, such as 0.5 V. The pH safety range is, for example, from pH 3 to 7. In certain embodiments, when the measured pH exceeds a pH safety range, for example the range from 3 to 7, the device switches the polarity for a short period of time to allow the pH to be reequilibrated.

According to a particular embodiment of the electroporation process, the process comprises a step of measuring the impedance of the skin. When the measured impedance exceeds an impedance safety range, the application of the current profile is reduced to a safety level to avoid an adverse event. The safety level may be less than 1 V, such as 0.5 V. The impedance safety range may be from 5 kΩ to 2 MΩ.

According to a particular embodiment of the electroporation process, the process comprises a step of measuring the temperature of the skin. When the measured temperature exceeds a temperature safety value, the application of the current profile is switched to a safety level, for example less than 1 V, such as 0.5 V. The temperature safety value can be chosen to be less than or equal to 43° C.

In a preferred embodiment of the electroporation process, the process comprises the steps of:
measuring the temperature of the skin,
measuring the impedance of the composition, and
measuring the pH of the composition.

The device is configured to process the results and to regulate the microcurrent and the polarity.

### Process

The process according to the invention may be cosmetic and non-therapeutic.

According to a particular embodiment of the electroporation process, the process comprises the transdermal delivery of a composition according to the invention including one or more hydroxy acid compounds.

According to a particular embodiment of the invention, the process for delivering the composition according to the invention comprising at least one hydroxy acid compound, in particular salicylic acid, through keratin materials, comprises the application of a specific pulsed electric current profile, from an electroporation device having at least one electrode and at least one counterelectrode, for a specific period of time sufficient to transdermally deliver the hydroxy acid compound(s), in particular salicylic acid, to a biological subject.

Thus, the diffusion through human keratin materials of the hydroxy acid compound(s), in particular salicylic acid, can be increased by applying the electroporation process according to the invention. Such an increase in the diffusion of the active agent makes it possible to optimize the amount of active agent required for the target treatments in the various layers of the keratin materials.

The electroporation treatment can be applied for a period of 60 s to 1000 s.

In particular, the electroporation treatment is applied for a period of 120 s to 480 s.

### Kit

According to a particular embodiment, the invention also proposes an electroporation kit comprising:
an electroporation composition comprising at least one hydroxy acid compound, in particular salicylic acid, and
an electroporation device for performing the electroporation process as described above.

The present invention also proposes an electroporation kit comprising:
an electroporation composition comprising at least one hydroxy acid compound, in particular salicylic acid, and
an electroporation device for delivering the electroporation composition through keratin materials, configured to apply a negative pulsed current profile.

Preferably, the present invention proposes a kit for performing the process as defined previously, including:
an electroporation device containing at least one electrode and at least one counterelectrode and arranged to subject keratin materials to pulse trains of a pulsed electric current, the pulse trains of the pulsed electric current repeating at a frequency (fₜᵣₐᵢₙ) of between 0.1 Hz and 1 kHz, the pulse trains each having a duration (Tₒₙ) of between 0.001 s and 2 s, and the pulsed electric current having a voltage amplitude ranging from 20 V to 250 V and a current ranging from 0.1 mA to 10 mA, and
a composition comprising at least one hydroxy acid compound, in particular salicylic acid.

The electroporation device and the composition may be packaged together, for example in the same package.

### Applications

In a preferred embodiment, the process, the composition and the kit according to the invention enable the treatment of wrinkles and the signs of ageing, and improve the softness, quality and appearance of the skin. It can thus be used to moisturize the skin, to improve the suppleness of the skin, to improve and/or reduce the microrelief of the skin, and also to combat the signs of skin ageing.

The process, the composition and the kit according to the invention are notably effective in delivering anti-ageing ingredients which impregnate the skin beyond the superficial levels, which may contribute to such effects. After ablative laser procedures or chemical peeling operations, for example, electroporation can also improve the healing process by achieving higher delivery of active agents. The rapid penetration that reaches a greater depth makes electroporation also effective for acne scars.

According to another particular embodiment, the process is used for minimizing the effects of age on the skin, and/or pigmentation, and/or the volume and/or sagging of wrinkles, and/or tone and/or spots, and/or for improving the firmness, and/or radiance, and/or softness of the skin.

### DESCRIPTION OF THE FIGURES

Other characteristics, variants and advantages of the invention will become apparent on reading the description of non-limiting illustrative examples thereof and on examining the appended drawings, in which:

### FIGURES

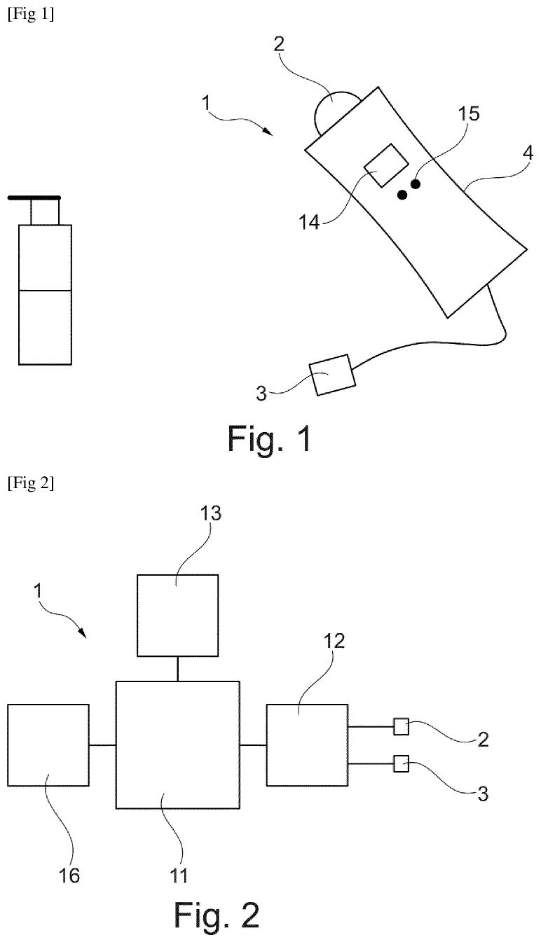

FIG. 1 is a schematic representation of an example of an electroporation device according to the invention,

FIG. 2 is a block diagram of the device,

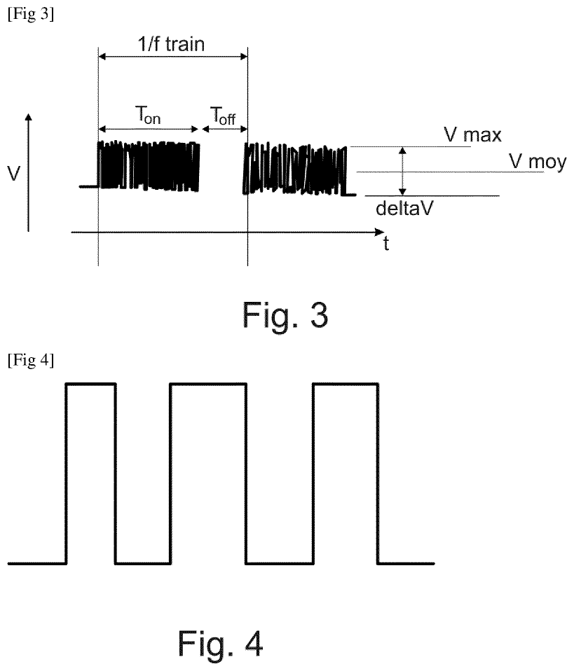

FIG. 3 illustrates the modulation of the output voltage,

FIG. 4 shows an elementary pattern of the output signal,

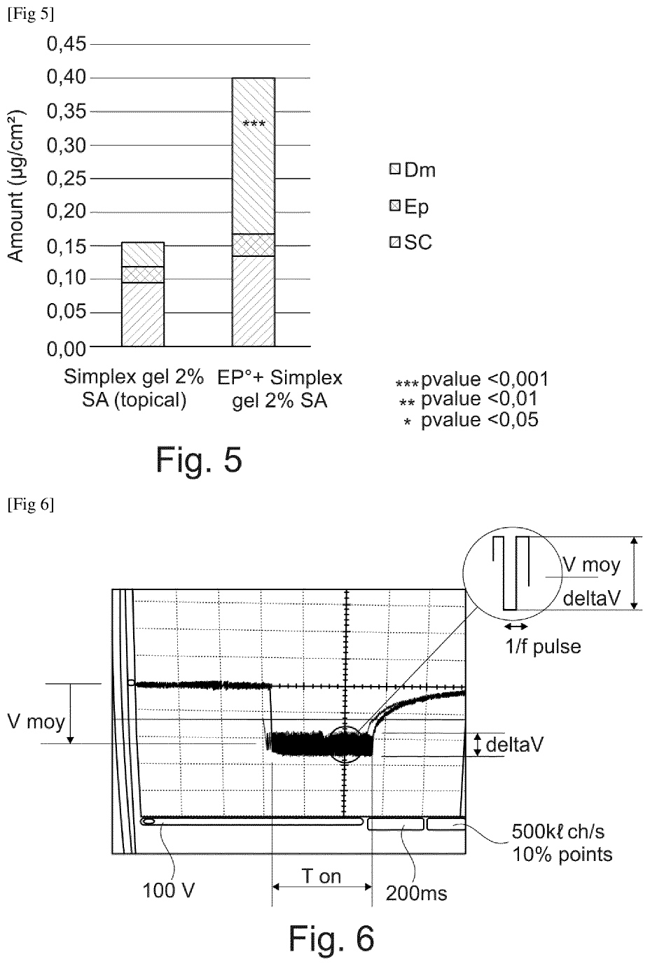

FIG. 5 is a diagram comparing the delivery of salicylic acid from a 2% simplex salicylic acid formulation topically into keratin materials and the delivery of salicylic acid from a 2% simplex salicylic acid formulation by electroporation, and

FIG. 6 illustrates the shape of the signal applied in one example.

FIG. 1 shows an example of a treatment device 1 according to the invention.

This device 1 has at least two electrical outputs between which an electrical signal is generated.

In the example considered, the device 1 includes at least one electrode 2, to be moved over the skin in the zones to be treated, and a counterelectrode 3 to be fixed onto the body, for example by means of a bracelet or an adhesive patch.

The electrode 2 can be carried, as shown in FIG. 1, by a case 4 which houses an electronic signal-generating circuit.

The electronic signal-generating circuit may include, as illustrated in FIG. 2, a central unit 11 with a microcontroller, for example of the ATMEGA type, which runs software for generating a signal of the desired shape, which is amplified by an amplifier 12 connected to the electrode 2 and to the counterelectrode 3.

The central unit 11 also manages the user interface 13, which includes, for example, a display 14 and one or more control buttons 15.

The electronic circuit can be powered by a battery 16 housed in the case 4, or as a variant by a mains adapter.

The output signal can be modulated.

The output signal may be a periodic signal, of given elemental pattern, which is modulated.

For example, the output signal may be a square wave periodic signal, as shown in FIG. 4, which is modulated according to a modulation profile as shown in FIG. 3, with a duration Tₒₙ during which the periodic signal of frequency fₚᵤₗₛₑ is present, thus generating a pulse train, followed by a duration T_{off} during which this signal is no longer present, this duration T_{off} preferably being a duration during which the voltage is zero between the electrode and the counterelectrode.

The duration Tₒₙ of a pulse train of the periodic signal is, for example, between 1 ms and 30 ms, better still between 2 ms and 10 ms, for example of the order of 5 microseconds when the device 1 is used in conjunction with a skin-peeling active agent, as detailed hereinbelow.

The frequency fₚᵤₗₛₑ of the pulses within the train is, for example, between 10 kHz and 50 kHz.

A duration Tₒₙ of less than or equal to 10 ms can reduce the risk of skin irritation or pain sensation or induction of muscle contractions, given that the nature of the applied field, in particular the geometry of the electrodes and their configuration (very close or very far apart, superficial versus through electric field when the electrodes are far apart), can lead to different perception thresholds.

A rest period T_{off} of greater than or equal to 100 ms allows the skin to recover a basal membrane potential between the application of the pulses.

The device 1 can be configured to generate an electrical signal between the electrode and the counterelectrode, for a predefined number of cycles, according to a pulse train of duration Tₒₙ, followed by a duration T_{off}, without pulses, for example by means of a pulse or pulse train counter, or for a predefined duration, by means of a timer.

The electrode 2 preferably has a rounded shape.

The skin contact area of the electrode 2 ranges, for example, from 2 cm² to 10 cm².

The electrical current density applied by the electrode 2 ranges, for example, from 0.1 mA/cm² to 0.5 mA/cm².

The voltage between the electrode 2 and the counterelectrode 3 preferably increases when a resistive load is connected thereto and when the resistance of the load increases. The amplitude is, for example, substantially zero for a zero resistance up to a resistance of 10 kOhms, then increases until it reaches about 200 V in open circuit.

The current (RMS) delivered by the device is preferably between 150 mA and 250 mA when it flows over a resistor whose value varies between 1 kOhm and 500 kOhms.

The device can be offered to the user with a composition P according to the invention to be applied to the skin, inside a common packaging, for instance a box.

The composition P according to the invention may be contained in any type of container, for example a container equipped with an applicator for applying the composition to the skin, or a pump bottle, among other possibilities.

Preferably, the applied voltage is unipolar, the voltage of the electrode 2 being, for example, either always positive or always negative, depending on the nature of the active agent(s) contained in the composition.

### Composition

An electroporation composition for use in an electroporation method as mentioned above is described.

A composition according to the invention has a viscosity such that the composition covers both the treated surface and the treatment electrode of the apparatus, allowing smooth, frictionless movement.

A composition according to the invention preferably has a viscosity of between 0.001 Pa·s and 0.5 Pa·s, better still between 0.005 Pa·s and 0.1 Pa·s, and even better still between 0.01 Pa·s and 0.05 Pa·s.

The viscosity of the composition is measured at room temperature (25° C.) using an RM 200 Plus rheometer (LamyRheology).

A composition according to the invention may be of gel type or is an emulsion, notably a water-in-oil or oil-in-water emulsion, and in particular an oil-in-water emulsion, also known as a direct emulsion.

According to a preferred embodiment, a composition according to the invention is of gel type, in particular of aqueous gel type.

A composition according to the invention may comprise an aqueous phase and/or an oily phase.

In certain embodiments of the electroporation composition, the composition has a pH ranging from 2 to 7.5, preferably from 3 to 6, and more preferentially from 3 to 5.

In certain embodiments, when the measured pH exceeds a pH safety range, for example the range from 3 to 6, the device switches the polarity for a short period of time to allow the pH to be reequilibrated.

As mentioned above, the electroporation method according to the present invention comprises a step of applying to human keratin materials a composition comprising a hydroxy acid compound.

### Hydroxy Acid Compounds

Hydroxy acid compound(s), in particular salicylic acid, are known to have one or more beneficial uses in humans, in particular as anti-ageing active agents.

The composition according to the invention comprises one or more hydroxy acids chosen from α-hydroxy acid compounds (AHA), β-hydroxy acid compounds (BHA), lipohydroxy acid compounds (LHA), polyhydroxy acid compounds (PHA), and mixtures thereof.

Preferably, the composition according to the invention comprises one or more hydroxy acids chosen from α-hydroxy acid compounds (AHA), β-hydroxy acid compounds (BHA), and mixtures thereof, which may notably be linear, branched or cyclic, and saturated or unsaturated.

### α-Hydroxy Acid Compounds

According to the present invention, the term “α-hydroxy acid” means a carboxylic acid containing at least one hydroxyl functional group occupying an a position on said acid (carbon adjacent to a carboxylic acid functional group). This acid may be present in the final composition in the form of the free acid and/or in the form of an associated salt thereof (salts with an organic base or an alkali metal, in particular), notably depending on the final pH imposed on the composition.

More particularly, the α-hydroxy acid compounds are chosen from glycolic acid, oxalic acid, lactic acid, 1-hydroxy-1-cyclopropanecarboxylic acid, 2-hydroxy-3-butenoic acid, 2-hydroxyisobutyric acid, 2-hydroxy-n-butyric acid, isoserine, glyceric acid, 2-hydroxy-3-methylbutyric acid, 2-hydroxy-2-methylbutyric acid, 2-hydroxyvaleric acid, 4-amino-2-hydroxybutyric acid, 1-hydroxycyclohexanecarboxylic acid, fumaric acid, dihydroxyfumaric acid, citramalic acid, tartaric acid, citric acid, 2-hydroxy-4-(methylthio)butyric acid, mandelic acid, 2-hydroxy-3-methylvaleric acid, glyoxyurea, β-imidazolelactic acid, 2-trifluoromethyl-2-hydroxypropionic acid, 2-hydroxybutanoic acid, 2-hydroxypentanoic acid, 2-hydroxyhexanoic acid, 2-hydroxyheptanoic acid, 2-hydroxyoctanoic acid, 2-hydroxynonanoic acid, 2-hydroxydecanoic acid, 2-hydroxyundecanoic acid, 2-hydroxydodecanoic acid, 2-hydroxytetradecanoic acid, 2-hydroxyhexadecanoic acid, 2-hydroxyoctadecanoic acid, 2-hydroxytetracosanoic acid, 2-hydroxyeicosanoic acid, hexahydromandelic acid, arabic acid, phenyllactic acid, hydroxyphenylglycine, 3-hydroxymandelic acid, 4-hydroxymandelic acid, methylacetic acid, L-arginic acid, 3-methoxymandelic acid, 4-methoxymandelic acid, 3-(4-hydroxyphenyl)lactic acid, tartronic acid, β-chlorolactic acid, 1-cyclopentanol-1-carboxylic acid, 1,2-dihydroxycyclobutanecarboxylic acid, 2-ethyl-2-hydroxybutyric acid, α-hydroxyisocaproic acid, α-hydroxycaproic acid, 2-hydroxy-3,3-dimethylbutyric acid, malic acid, hydroxytartronic acid, gluconic acid, glucuronic acid, pyruvic acid, galacturonic acid, aleuritic acid, ribonic acid, lactamide, N-methyllactamide, N-ethyllactamide, N,N-dimethyllactamide, N-2-hydroxyethyllactamide, salts thereof and mixtures thereof. It is also possible to use mixtures of these various acids.

More particularly, mention may be made of the following examples of α-hydroxy acids: citric acid, oxalic acid, lactic acid, glycolic acid, glyceric acid, tartaric acid, malic acid, mandelic acid, methylacetic acid, glucuronic acid, pyruvic acid, phenyllactic acid, arabic acid, gluconic acid, galacturonic acid, hydroxytartronic acid, lactamide, N-methyllactamide, N-ethyllactamide, N-2-hydroxyethyllactamide, and mixtures thereof.

In particular, the preferred compounds are chosen from glycolic acid, oxalic acid, lactic acid, L-lactic acid, DL-lactic acid, D-lactic acid, malic acid, tartaric acid, DL-glyceric acid, arabic acid, gluconic acid, hydroxytartronic acid, lactamide, N-methyllactamide, N-ethyllactamide, N-2-hydroxyethyllactamide, and mixtures thereof.

According to a preferred embodiment, the α-hydroxy acid is chosen from citric acid, malic acid, tartaric acid, lactic acid and salts thereof more particularly, the α-hydroxy acid is chosen from citric acid, lactic acid, salts thereof and mixtures thereof.

### β-Hydroxy Acid Compounds

According to the present invention, the term “β-hydroxy acid” means a carboxylic acid containing at least one hydroxyl functional group occupying a R position on said acid. This acid may be present in the final composition in the form of the free acid and/or in the form of an associated salt thereof (salts with an organic base or an alkali metal, in particular), notably depending on the final pH imposed on the composition.

More particularly, examples of β-hydroxy acid compounds that may be mentioned include: salicylic acid and derivatives thereof, in particular the derivatives of formula (I) below or a salt of such a derivative:

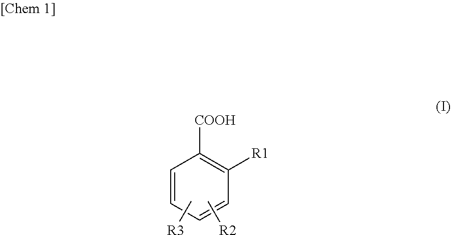

in which:
the radical R₁ represents a hydroxyl group or an ester of formula: —O—CO—R₄, in which R₄ is a saturated or unsaturated aliphatic group containing from 1 to 26 carbon atoms, and preferably from 1 to 18 carbon atoms, or an amine or thiol group which may be substituted with an alkyl group containing from 1 to 18 carbon atoms, and preferably from 1 to 12 carbon atoms,
R₂ and R₃, independently of each other, are located in position 3, 4, 5 or 6 on the benzene nucleus and, independently of each other, represent a hydrogen atom or a group —(O)ₙ—(CO)ₘ—R₅, where n and m, independently of each other, are each an integer equal to 0 or 1, with the proviso that R₂ and R₃ do not simultaneously represent a hydrogen atom; R₅ represents a hydrogen atom, a linear, branched or cyclic saturated aliphatic group containing from 1 to 18 carbon atoms, an unsaturated group containing from 3 to 18 carbon atoms, bearing one to nine conjugated or non-conjugated double bonds, the groups being optionally substituted with at least one substituent chosen from halogen atoms (fluoro, chlorine, bromine or iodine), trifluoromethyl groups, hydroxyl groups in free form or esterified with an acid containing from 1 to 6 carbon atoms, or carboxyl groups, which are free or esterified with a lower alcohol containing from 1 to 6 carbon atoms.

Preferably, the salicylic acid derivative of formula (I) is such that R₁ represents a hydroxyl group, R₂ represents a hydrogen atom, and R₃ is in position 5 of the benzene nucleus and represents a group —CO—R₅ in which R₅ represents a saturated aliphatic group containing from 3 to 15 carbon atoms.

According to a preferred embodiment, the salicylic acid derivative of formula (I) is chosen from 5-n-octanoylsalicylic acid, 5-n-decanoylsalicylic acid, 5-n-dodecanoylsalicylic acid, 5-n-octylsalicylic acid, 5-n-heptyloxysalicylic acid, 4-n-heptyloxysalicylic acid, 5-tert-octylsalicylic acid 3-tert-butyl-5-methylsalicylic acid, 3-tert-butyl-6-methylsalicylic acid, 3,5-diisopropylsalicylic acid, 5-butoxysalicylic acid, 5-octyloxysalicylic acid, propanoyl-5-salicylic acid, 5-n-hexadecanoyl-5-salicylic acid, 5-n-oleoylsalicylic acid and 5-benzoylsalicylic acid, the monovalent and divalent salts thereof and mixtures thereof.

Preferably, the composition comprises at least one β-hydroxy acid compound, preferably salicylic acid.

The hydroxy acid compound(s), in particular the β-hydroxy acid compound(s), preferably salicylic acid, may be present in the composition according to the present invention in a content of between 0.1% and 15%, preferably between 0.5% and 10%, and more preferentially between 1% and 5% by weight, relative to the total weight of the composition.

### Aqueous Phase

A composition according to the invention preferably comprises an aqueous phase.

According to a preferred embodiment, the composition according to the present invention has a water content of between 20% and 100% by weight, in particular between 40% and 100% by weight and preferably between 60% and 100% by weight relative to the total weight of the composition.

According to a preferred embodiment, the aqueous phase of a composition according to the invention also comprises at least one alcohol, notably chosen from monoalcohols and polyols, in particular chosen from glycols.

Thus, the composition may comprise at least one monoalcohol, preferably chosen from ethanol and isopropanol, and more preferentially at least ethanol.

Preferably, the composition comprises glycerol.

Preferably, the mass concentrations of monoalcohol(s) are between 0.1% and 30%, and preferably between 5% and 15% by weight, relative to the total weight of said composition.

The composition may also comprise a polyol chosen from propylene glycol, propanediol, dipropylene glycol, butylene glycol, pentylene glycol, hexylene glycol, caprylyl glycol, glycerol and sugars such as sorbitol, (poly)alkylene glycols, and mixtures thereof.

Preferably, the composition may comprise a polyol chosen from propanediol and propylene glycol, and mixtures thereof.

According to one embodiment, the mass concentrations of polyol range from 0.01% to 40% by weight, relative to the total weight of said composition.

Preferably, the mass concentrations of polyol range from 0.1% to 30% by weight and preferably from 5% to 15% by weight relative to the total weight of said composition.

### Thickener/Gelling Agent

According to a preferred embodiment, a composition according to the present invention may comprise a thickener/gelling agent.

Thus, depending on the viscosity of the composition that it is desired to obtain, one or more thickeners/gelling agents, which are notably hydrophilic, i.e. soluble or water-dispersible, may be incorporated into the composition.

Examples of hydrophilic gelling agents that may be mentioned include modified or unmodified carboxyvinyl polymers, such as the products sold under the names Carbopol (CTFA name: Carbomer) and Pemulen (CTFA name: Acrylates/C₁₀₋₃₀ alkyl acrylate crosspolymer) by the company Goodrich, polyacrylamides, optionally crosslinked and/or neutralized 2-acrylamido-2-methylpropanesulfonic acid polymers and copolymers, for instance the poly(2-acrylamido-2-methylpropanesulfonic acid) sold by the company Hoechst under the name Hostacerin AMPS® (CTFA name: Ammonium polyacryldimethyltauramide), crosslinked anionic copolymers of acrylamide and of AMPS®, which are in the form of a water-in-oil emulsion, such as those sold under the name Sepigel 305 (CTFA name: Polyacrylamide/C₁₃₋₁₄ Isoparaffin/Laureth-7) and under the name Simulgel 600 (CTFA name: Acrylamide/Sodium acryloyldimethyltaurate copolymer/isohexadecane/Polysorbate 80) by the company SEPPIC, polysaccharide biopolymers such as modified celluloses, carrageenans, notably kappa-carrageenan, gellan gum, agar-agar, xanthan gum, alginate-based compounds, in particular sodium alginate, scleroglucan gum, guar gum, inulin and pullulan, cassia gum, karaya gum, konjac gum, gum tragacanth, tara gum, acacia gum or gum arabic, and mixtures thereof. The amount of gelling agents depends on the desired objective.

According to a preferred embodiment, a composition according to the present invention comprises a polysaccharide, notably chosen from a cellulose or a derivative thereof, in particular cellulose ethers or esters.

The cellulose derivatives may be anionic, cationic, amphoteric or nonionic. Among these derivatives, cellulose ethers, cellulose esters and cellulose ether esters are distinguished.

Among the nonionic cellulose ethers, mention may notably be made of alkylcelluloses such as methylcelluloses and ethylcelluloses, hydroxyalkylcelluloses such as hydroxymethylcelluloses, hydroxyethylcelluloses and hydroxypropylcelluloses, and mixed hydroxyalkylalkylcelluloses such as hydroxypropylmethylcelluloses, hydroxyethylmethylcelluloses, hydroxyethylethylcelluloses and hydroxybutylmethylcelluloses.

The celluloses and derivatives are represented, for example, by the products sold under the names Avicel® (microcrystalline cellulose, MCC) by the company FMC Biopolymers, under the name Cekol (carboxymethylcellulose) by the company Noviant (CP-Kelco), under the name Akucell AF (sodium carboxymethylcellulose) by the company AkzoNobel, under the name Methocel™ (cellulose ethers) and Ethocel™ (ethylcellulose) by the company Dow, and under the names Aqualon® (carboxymethylcellulose and sodium carboxymethylcellulose), Benecel® (methylcellulose), Blanose™ (carboxymethylcellulose), Culminal® (methylcellulose, hydroxypropylmethylcellulose), Klucel® (hydroxypropylcellulose), Polysurf® (cetylhydroxyethylcellulose) and Natrosol® CS (hydroxyethylcellulose) by the company Hercules Aqualon.

According to a preferred embodiment, a composition according to the invention comprises at least one hydroxypropylmethylcellulose and/or poly(2-acrylamido-2-methylpropanesulfonic acid).

According to a preferred embodiment, a composition according to the invention comprises at least one hydroxypropylmethylcellulose.

According to a preferred embodiment, a composition according to the invention comprises at least one poly(2-acrylamido-2-methylpropanesulfonic acid).

In particular, the amount of thickeners/gelling agents ranges, for example, from 0.01% to 10%, notably from 0.1% to 5% by weight, preferably from 0.1% to 3% by weight, relative to the total weight of the composition.

### Oily Phase

As mentioned above, a composition according to the invention may comprise an oily phase. When the composition used according to the invention comprises an oily phase, it preferably contains at least one oil, notably a cosmetic oil. It may also contain other fatty substances. Among the oils that may be used in the composition of the invention, mention may be made of hydrocarbon-based oils of animal origin, hydrocarbon-based oils of plant origin, synthetic esters and ethers, notably of fatty acids, hydroxylated esters, polyol esters, pentaerythritol esters, linear or branched hydrocarbons of plant, mineral or synthetic origin, fatty alcohols and fatty acids containing from 8 to 26 carbon atoms, silicone-based and/or partially hydrogenated fluoro oils, silicone oils and mixtures thereof.

The other fatty substances that may be present in the oily phase are, for example, fatty acids comprising from 8 to 30 carbon atoms, for example stearic acid, lauric acid, palmitic acid and oleic acid; waxes, for example lanolin wax, beeswax, carnauba wax or candelilla wax, paraffin wax, lignite wax or microcrystalline waxes, ceresin or ozokerite, and synthetic waxes, for example polyethylene waxes and Fischer-Tropsch waxes; silicone resins such as trifluoromethyl (C₁-C₄)alkyl dimethicone and trifluoropropyl dimethicone; and silicone elastomers.

These fatty substances may be chosen in a varied manner by a person skilled in the art in order to prepare a composition having the desired properties, for example in terms of consistency or texture.

In particular, the amount of oily phase may range, for example, from 0% to 30%, and in particular from 0.1% to 15% by weight, relative to the total weight of the composition.

### Surfactant/Emulsifier

According to one embodiment, the composition according to the invention may comprise at least one emulsifier.

Among the emulsifiers that may be used in the emulsions, examples that may be mentioned include nonionic emulsifiers such as oxyalkylenated (more particularly polyoxyethylenated) fatty acid esters of glycerol; oxyalkylenated fatty acid esters of sorbitan; oxyalkylenated (oxyethylenated and/or oxypropylenated) fatty acid esters; oxyalkylenated (oxyethylenated and/or oxypropylenated) fatty alcohol ethers; sugar esters such as sucrose stearate; and mixtures thereof, such as the mixture of glyceryl stearate and PEG-40 stearate.

Mention may also be made of fatty alcohol/alkylpolyglycoside emulsifying mixtures as described in patent applications WO 92/06778, WO 95/13863 and WO 98/47610, for example the commercial products sold by the company SEPPIC under the name Montanov®.

Among the emulsifiers that may be used in the compositions according to the invention, examples that may be mentioned include alkyl dimethicone copolyols, for example Cetyl PEG/PPG-10/1 Dimethicone and more particularly the mixture Cetyl PEG/PPG-10/1 Dimethicone and Dimethicone (INCI name), such as the product sold under the brand name Abil EM90 by the company Goldschmidt, or the mixture (Polyglyceryl-4 stearate and Cetyl PEG/PPG-10 (and) Dimethicone (and) Hexyl Laurate), such as the product sold under the brand name Abil WE09 by the same company.

Mention may also be made of dimethicone copolyols, for example PEG-18/PPG-18 Dimethicone and more particularly the mixture Cyclopentasiloxane (and) PEG-18/PPG-18 Dimethicone (INCI name), such as the product sold by the company Dow Corning under the brand name Silicone DC5225 C or KF-6040 from the company Shin-Etsu.

Mention may also be made of nonionic emulsifiers derived from fatty acids and polyols, alkylpolyglycosides (APGs) and sugar esters, and also mixtures thereof.

Mention may also be made of succinic-terminated polyolefins, for instance esterified succinic-terminated polyisobutylenes and salts thereof, notably the diethanolamine salts, such as the products sold under the names Lubrizol 2724, Lubrizol 2722 and Lubrizol 5603 by the company Lubrizol or the commercial product Chemcinnate 2000.

According to a preferred embodiment, the composition according to the invention comprises at least one nonionic emulsifier.

The total amount of emulsifiers in the composition will preferably be, in the composition according to the invention, in active material contents ranging from 0% to 8% by weight and more particularly from 0.5% to 6% by weight relative to the total weight of the composition.

### Base

In embodiments of the electroporation composition, the composition may also comprise at least one base.

The base may be chosen from mineral bases, for instance alkali metal hydroxides, sodium hydroxide, potassium hydroxide, ammonium hydroxides, aqueous ammonia, organic bases, for example monoethanolamine, diethanolamine, triethanolamine, triisopropylamine, tris[(2-hydroxy)-1-propyl]amine, N,N-dimethylethanolamine, 2-amino-2-methyl-1-propanol, 2-amino-2-methyl-1,3-propanediol, triethylamine, dimethylaminopropylamine and amphoteric bases (i.e. bases containing both anionic and cationic functional groups), such as primary, secondary, tertiary or cyclic organic amines, and amino acids. Examples of amphoteric bases that may be mentioned include glycine, lysine, arginine, taurine, histidine, alanine, valine, cysteine, trihydroxymethylaminomethane (TRISTA), triethanolamine, and any mixture thereof.

According to a particular embodiment, the base of the composition is chosen from sodium hydroxide, potassium hydroxide, ammonium hydroxides, ammonia, monoethanolamine, diethanolamine, triethanolamine, tromethamine, and mixtures thereof.

According to a particular embodiment, the bases of the composition are triethanolamine and sodium hydroxide.

According to a particular embodiment, the base of the composition according to the invention is present in a weight concentration of less than 0.5% by weight, or even less than 0.3% by weight, relative to the total weight of the composition.

The total amount of base(s) in the composition will preferably be, in the composition according to the invention, in contents ranging from 0.1% to 5% by weight and more particularly from 0.2% to 3% by weight, relative to the total weight of the composition.

### Other Additives

The cosmetic composition may also comprise at least one additive chosen from adjuvants typical of the cosmetic field, such as hydrophilic or lipophilic gelling agents, water-soluble or liposoluble active agents, for example anti-ageing active agents, film-forming polymers, preserving agents, sequestrants, antioxidants, solvents, fragrances, odour absorbers, pH correctors, and mixtures thereof.

According to one embodiment, the composition according to the invention may also comprise at least one preserving agent, preferably disodium EDTA and/or phenoxyethanol.

According to a preferred embodiment, the composition according to the invention is free of preserving agents. Such an embodiment is notably advantageous when the pH of the composition is low, for example between 3.5 and 4.5.

A person skilled in the art will take care to select the optional additional adjuvants and/or the amount thereof so that the advantageous properties of the composition are not, or are not substantially, adversely affected by the envisaged addition.

### Physiologically Acceptable Medium

The composition, notably the cosmetic composition, according to the invention comprises a physiologically acceptable medium.

For the purposes of the present invention, the term “physiologically acceptable medium” is intended to refer to a medium that is suitable for the topical administration of a composition. A physiologically acceptable medium generally has no unpleasant odour or appearance, and is entirely compatible with topical administration. In the present case, when the composition is intended for topical administration, i.e. by application to the surface of the keratin material under consideration, such a medium is notably considered to be physiologically acceptable when it does not cause stinging, tautness or redness that is unacceptable to the user.

In particular, the composition is suited to topical application, i.e. application to the surface of the skin, the scalp and/or the mucous membrane under consideration. Thus, the physiologically acceptable medium is preferably a cosmetically or dermatologically acceptable medium, i.e. a medium that has no unpleasant odour, colour or appearance, and that does not cause the user any unacceptable stinging, tautness or redness.

The composition may then comprise any constituent normally used in the intended application.

Needless to say, a person skilled in the art will take care to select the optional additional compound(s), and/or the amount thereof, such that the advantageous properties of the compounds according to the invention are not, or are not substantially, adversely affected by the envisaged addition.

Unless otherwise indicated, the amounts indicated are expressed as mass percentages.

### Example

To test the effect of the device, a sample of human skin is peripherally attached to a support, with the counterelectrode of the electroporation device placed between the inner face of the skin and the support. The electrode of the electroporation device is placed in contact with the outer face of the skin sample. The case of the electroporation device is immobilized by a support. The support is placed on an orbital shaker, so that the electrode moves over the surface of the skin.

The device used in this example is the one sold by the company Fusion under the name Fusion Meso Xpert, after deactivation of the LED lighting.

The frequency (fₜᵣₐᵢₙ) with which the pulse trains follow each other is 0.4 Hz, the duration Tₒₙ ranges from 0.5 to 2 seconds, and the chosen polarity is negative, i.e. the electrode is at a negative potential with respect to the counterelectrode. The apparatus is used in the highest energy mode. At the voltage plateau of mean value Vₘₑₐₙ during the pulse train of duration Tₒₙ, the signal shows a variation at a frequency fₚᵤₗₛₑ of the voltage around the mean (negative) voltage Vₘₑₐₙ, as shown in FIG. 6. The pulses of frequency fₚᵤₗₛₑ are, for example, of maximum amplitude deltaV, within the pulse train, this amplitude deltaV being less than or equal to 30% of the voltage Vₘₑₐₙ, as an absolute value.

It is seen that in the absence of a pulse train, i.e. during T_{off}, the voltage is zero.

This corresponds to a Tₒₙ/(Tₒₙ+T_{off}) duty cycle of substantially 80%, for example.

In open circuit, the voltage between the electrode and the counterelectrode is about 200 V.

On the skin, the voltage between the electrode and the counterelectrode is about 40 V.

The cosmetic compositions are applied before the apparatus is put in place.

Three gel-type cosmetic compositions are prepared according to the following protocol: Composition 1: Microbiologically clean deionized water is poured into the formulation beaker as the first phase. Propylene glycol, 1,3-propanediol, triethanolamine, phenoxyethanol and EDTA are successively added. Once the mixture is homogenized, salicylic acid powder is added and the whole mixture is then mixed with a Rayneri blender. The gelling agent AMPS is gradually added, followed by the denatured alcohol last, so as to avoid evaporation, with stirring until a perfectly homogeneous gel is obtained, which is then debubbled.

Compositions 2 and 3: Microbiologically clean deionized water is poured into the formulation beaker as the first phase. Propylene glycol, 1,3-propanediol and phenoxyethanol are successively added. Once the mixture is homogenized, salicylic acid powder is added and the whole mixture is then mixed with a Rayneri blender. The gelling agent hydroxypropylmethylcellulose is added and then the denatured alcohol is added last, so as to avoid evaporation, with stirring. The pH is adjusted with sodium hydroxide until a perfectly homogeneous gel is obtained, which is then debubbled.

The compositions have the following formulations (the amounts are expressed on a weight basis, relative to the total weight of the composition):

TABLE 1

| Compounds | | Formulation 1 | Formulation 2 | Formulation 3 |
|---|---|---|---|---|
| (INCI name) | Commercial reference | (% by weight) | (% by weight) | (% by weight) |
| Sodium Hydroxide | SODIUM HYDROXIDE 30% | / | 0.57 | 0.29 |
| Disodium EDTA | Disodium EDTA | 0.05 | / | / |
| Triethanolamine | Triethanolamine 99% | 2.3 | / | / |
| Salicylic Acid | SALICYLIC ACID | 2 | 2 | 1 |
| Phenoxyethanol | SEPICIDE LD | 0.5 | 0.5 | 5 |
| Hydroxypropyl | METHOCEL F 4 M | / | 1 | 1 |
| Methylcellulose | PERSONAL CARE GRADE | | | |
| Ammonium | HOSTACERIN AMPS | 2 | / | / |
| Polyacryloyldimethyl | | | | |
| Taurate | | | | |
| Alcohol denat. | DENATURED ETHYL | 10 | 10 | 10 |
| | ALCOHOL | | | |
| Propylene Glycol | MONOPROPYLENE | 5 | 5 | 5 |
| | GLYCOL UPS | | | |
| Propanediol | ACTIVONOL-3 | 5 | 5 | 5 |
| Water | MICROBIOLOGICALLY | qs 100 | qs 100 | qs 100 |
| | CLEAN DEIONIZED | | | |
| | WATER | | | |

### Comparative Example

Firstly, compositions 1 to 3 are applied topically, and secondly, the application of compositions 1 to 3 is combined with electroporation.

### Results

The skin is removed from the experimental system and then analysed to determine the amount of salicylic acid that has penetrated.

The results are given in FIG. 5.

It is shown that, by means of the process according to the invention, the amount of salicylic acid that has penetrated is much higher, compared with the topical application of an identical composition.

In the entire description, including the claims, the expression “comprising one” is to be understood as being synonymous with “comprising at least one”, unless the contrary is specified.

The expressions “between . . . and . . . ”, “comprises from . . . to . . . ”, “formed from . . . to . . . ” and “ranging from . . . to . . . ” should be understood as being inclusive of the limits, unless otherwise specified.

1. Cosmetic and non-therapeutic electroporation process for delivering a composition through human keratin materials, the composition comprising at least one hydroxy acid compound, the electroporation process comprising the exposure of the composition in contact with said keratin materials to pulse trains of a pulsed electric current, the pulsed electric current being provided by an electroporation device (1) having at least one electrode (2) and at least one counterelectrode (3), the pulse trains of the pulsed electric current repeating at a frequency (fₜᵣₐᵢₙ) of between 0.1 Hz and 1 kHz, the pulse trains each having a duration (Tₒₙ) of between 0.001 s and 2 s, and the pulsed electric current having a voltage amplitude ranging from 20 V to 250 V, and a current ranging from 0.1 mA to 10 mA.

2. Electroporation process according to claim 1, the maximum voltage between the electrode (2) and the counterelectrode (3) in open circuit ranging from 20 V to 250 V.

3. Electroporation process according to claim 1, the pulsed electric current having a duty cycle (Tₒₙ/(Tₒₙ+T_{off})) ranging from 20% to 90%, where Tₒₙ denotes the duration of a pulse train and T_{off} denotes the interval between two successive pulse trains.

4. Electroporation process according to claim 1, the pulse trains repeating at a frequency fₜᵣₐᵢₙ ranging from 0.1 Hz to 1 kHz, the frequency (fₚᵤₗₛₑ) of the pulses within the pulse train being between 5 kHz and 100 kHz.

5. Electroporation process according to claim 1, the pulse trains each having a duration (Tₒₙ) ranging from 0.01 s to 3 s.

6. Electroporation process according to claim 1, the pulse trains being separated by a pulse-free interval (T_{off}) ranging from 0.01 s to 3 s.

7. Electroporation process according to claim 1, the surface area of the electrode(s) (2) in contact with the keratin materials ranging from 2 to 20 cm².

8. Electroporation process according to claim 1, the counterelectrode being static and having a surface area in contact with the human keratin materials that is greater than that of the electrode, which is moved in contact with the keratin materials.

9. Electroporation process according to claim 1, consisting in manually moving the electrode(s) (2) of the electroporation device over the keratin materials while subjecting said keratin materials to said pulse trains of pulsed electric current.

10. Electroporation process according to claim 1, the pulsed electric current having a general square or rectangular waveform.

11. Electroporation process according to claim 1, the electroporation device (1) being hand-held.

12. Electroporation process according to claim 1, comprising topical application of the composition to the zone to be treated before applying the pulsed electric current and/or during said application.

13. Electroporation process according to claim 1, the electrode(s) (2) having a rounded surface for contact with the keratin materials.

14. Electroporation process according to claim 1, the composition comprising one or more hydroxy acids chosen from α-hydroxy acid compounds (AHA), β-hydroxy acid compounds (BHA), lipohydroxy acid compounds (LHA), polyhydroxy acid compounds (PHA), and mixtures thereof which may notably be linear, branched or cyclic, and saturated or unsaturated.

15. Electroporation process according to claim 1, the composition comprising at least one β-hydroxy acid compound.

16. Electroporation process according to claim 1, the composition comprising at least one β-hydroxy acid compound, in a content of between 0.1% and 15%, relative to the total weight of the composition.

17. Electroporation process according to claim 1, composition having a water content of between 20% and 100% by weight, relative to the total weight of the composition.

18. Electroporation process according to claim 1, the composition comprising at least one alcohol.

19. Electroporation process according to claim 1, the composition comprising glycerol.

20. Electroporation process according to claim 1, the composition comprising a thickener/gelling agent.

21. Electroporation process according to claim 1, the composition having a pH ranging from 2 to 7.5.

22. Electroporation process according to claim 1, the composition being free of preserving agents.

23. Kit for performing the process according to claim 1, including:
an electroporation device containing at least one electrode (2) and at least one counterelectrode (3) and arranged to subject keratin materials to pulse trains of a pulsed electric current, the pulse trains of the pulsed electric current repeating at a frequency (fₜᵣₐᵢₙ) of between 0.1 Hz and 1 kHz, the pulse trains each having a duration (Tₒₙ) of between 0.001 s and 2 s, and the pulsed electric current having a voltage amplitude ranging from 20 V to 250 V and a current ranging from 0.1 mA to 10 mA, and
a composition comprising at least one hydroxy acid compound.
